(19) United States
(12) Patent Application Publication
(10) Pub. No.: US 2025/0127810 A1
(43) Pub. Date: Apr. 24, 2025

(54) METHOD AND APPARATUS FOR PREPARING PLASMA ENRICHED IN IL-1RA

(71) Applicant: ESTAR TECHNOLOGIES LTD., Holon (IL)

(72) Inventor: Aaron ESTERON, Holon (IL)

(73) Assignee: ESTAR TECHNOLOGIES LTD., Holon (IL)

(21) Appl. No.: 18/684,934

(22) PCT Filed: Aug. 23, 2022
(86) PCT No.: PCT/IL2022/050924

§ 371 Date: Feb. 20, 2024

Related U.S. Application Data
(60) Provisional application No. 63/236,130, filed on Aug. 23, 2021.

(51) Int. Cl. A61K 35/16; A61K 38/17; B01D 21/26; B01D 29/01; B01D 29/11; G01N 33/68

(52) U.S. Cl. CPC A61K 35/16; A61K 38/1793; B01D 21/262; B01D 29/01; B01D 29/11; G01N 33/6869; G01N 2333/7155

(57) ABSTRACT

Methods and apparatus are disclosed for preparing plasma enriched with Interleukin Receptor 1 antagonist (“IL-1Ra”) using glass beads or polyacrylamide microbeads and recovering the plasma enriched in IL-1Ra with a filter.

### FIELD OF THE INVENTION

The present invention relates to methods and apparatus for preparing plasma enriched with Interleukin Receptor 1 antagonist (“IL-1Ra”) using glass beads or polyacrylamide microbeads. Embodiments of the invention relate to preparation of platelet rich plasma (PRP) compositions enriched in IL-1Ra.

### BACKGROUND OF THE INVENTION

U.S. Pat. Nos. 9,962,480 and 10,617,812, by the inventor herein, include embodiments directed to systems and methods for obtaining cellular samples enriched with cells, such as a platelet rich plasma (PRP), including separations using centrifugation of a blood sample in a centrifugation tube using a density separation medium and separation of cellular components using various filter systems. The disclosure of these patents is incorporated by reference.

U.S. Pat. No. 8,734,373, by the inventor herein, describes devices and methods for preparing PRP, including using an elongated filter device adapted for tight fitting into a test tube for separation of cellular components from a plasma fraction. The disclosure of this patent is also incorporated by reference as it relates to a filter adapted to be inserted into a collection tube.

U.S. Pat. Nos. 10,167,310 and 10,519,196, also by the inventor herein, include embodiments directed to systems and methods for obtaining a plasma fraction enhanced in Interleukin-1 Receptor Antagonist (IL-1Ra). In embodiments, the disclosed methods involve subjecting a blood sample in a blood collection tube to centrifugation and incubating the plasma fraction to enrich the fraction in IL-1Ra. The disclosure of these patents is likewise incorporated by reference.

There continues to be a need in the art for methods and devices for obtaining a plasma sample enriched in IL-1Ra, with enhanced recovery of IL-1Ra, and to methods for providing PRP enriched in IL-1Ra with greater efficiency.

### SUMMARY OF THE INVENTION

Some aspects of the invention pertain to simple and effective methods for obtaining a plasma sample enriched in IL-1Ra, and to methods for providing PRP enriched in IL-1Ra.

In one aspect, the invention is a method of preparing a plasma composition enriched in IL-1Ra, comprising: collecting a PRP fraction in a collection vessel or collection tube containing polymeric microbeads; mixing the polymeric microbeads with the PRP fraction to form a homogeneous mixture of microbeads and PRP; allowing the homogeneous mixture to sit for a predetermined period of time; passing the mixture through a filter (and/or centrifuging the vessel or tube containing the mixture) to separate the microbeads, along with cells and/or cellular fractions of the plasma, and to obtain the plasma composition enriched in IL-1Ra.

In embodiments, the recovery of IL-1Ra (defined as the IL-1 Ra mass in the final product after treatment divided by the IL-1Ra mass in the baseline PRP) is in a range 2.2 to 21 fold, including intermediate values, such as greater than 5 fold, and greater than 10 fold. As an increase in concentration (picograms/ml) the techniques of the invention may yield an increase in concentration of 2.8 to 25.8 fold over a baseline.

In another aspect, the invention is embodied in a method of preparing a plasma composition enriched in IL-1Ra, comprising: collecting a whole blood sample in a centrifugation tube equipped with glass beads; contacting the whole blood sample with the surface of the beads for a predetermined period of time (for example, for i) 4 to 24 hours at a temperature of 30-40° C. or ii) 5 minutes to 1 hour at room temperature); centrifuging the whole blood sample to obtain IL-1Ra enriched plasma; passing IL-1Ra enriched plasma through a filter to obtain the plasma composition enriched in IL-1Ra.

In embodiments, the recovery of IL-1 Ra (as compared to baseline) using the glass beads is greater than the recovery without the glass beads by a factor of 1.2, 1.3, 1.4 or more. In embodiments, the glass beads may be used in combination with polymeric microbeads and in combination with a separation medium (such as a gel), with or without the polymeric microbeads.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts a process sequence in which PRP is enriched in IL-1Ra by incubating the PRP on polymeric microbeads followed by recovering IL-1Ra-enriched PRP with a sleeve-filter according to an embodiment of the invention.

FIG. 2 depicts a process sequence in which whole blood is incubated with glass beads, followed by centrifugation and recovery of IL-1Ra-enriched plasma according to an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

A vessel or tube for use with the invention may be vacuum or non-vacuum, made of glass or plastic. In embodiments, a collection tube used with the invention is made of durable plastic such as polyacrylate (PA) or polyethylene terephthalate (PET), polyethylene terephthalate glycol (PETG) or polyethylene naphthalate (PEN). In other embodiments, the tube or vessel may be glass or coated glass.

In embodiments, the vessel or tube is provided with a separation medium, such as a gel, with a density in a range of 1.04-1.08 g/ml, and in embodiments between 1.073-1.078 g/ml. In embodiments, an anticoagulant is provided in the tube or vessel, such as heparin salts, citrate/citric (e.g., sodium citrate/citric acid), citrate/citric-dextrose (acid-citrate-dextrose ACD), citrate-dextrose-phosphate, oxalate, and oxalate-fluoride salt. In embodiments the vessel or tube is provided without gel. In embodiments, the vessel or tube is provided without anticoagulant.

The vessel or tube may be preinserted with glass beads and/or polymer microbeads with high rate of water absorption, such as polyacrylamide (PAA) microbeads. Alternatively, glass beads may be used, of a type conventionally used for incubation of cellular samples. Examples of both systems are provided below with sometimes surprisingly different result depending on the combination of process steps.

The above vessel or tube may be filled with the 10-100 ml of whole blood, for example 10-50 ml of whole blood. Alternatively, the glass beads or the polymer microbeads may be preinserted in a second vessel or tube which is then used to collect the plasma or the PRP fraction produced in a separate vessel or tube.

Where anticoagulant and glass beads are preinserted into a collection tube, the tube may be either incubated for 4-24 hours or placed in an ambient temperature for zero to 1 hour, followed by centrifugation at 1000 g to 3000 g, for example at 1500 g where a fraction of plasma enriched in IL-1Ra may be separated by sleeve filter as described in the aforesaid U.S. Pat. No. 10,519,196 or directly transferred to syringe or to a syringe via disc filter of 0.22-0.45 micron to separate cells and cells residue and microbeads and to enable the plasma with the enriched IL-1 Ra to be stored at appropriate temperature, for example −5 to −20° C. which allows dosage forms of the IL-1Ra to be stored and prepared for use. The above filtration procedure may be processed after a second centrifugation, for example at 2000-4000 g for 5-10 minutes to deposit cells, cells fractions and microbeads.

Where the first vessel or collection tube is provided without glass beads or polymer microbeads, a blood sample may be centrifuged for several minutes, for example 5-10 minutes at 1000-3000 g, for example at 1500 g to obtain separated plasma with an upper fraction of platelet poor plasma (PPP) and lower fraction (still above gel) of PRP enriched in monocytes and lymphocytes but depleted in erythrocytes and granulocytes due to the specific properties of the gel that act as a separator. PPP is removed, for example 50%-75% of the whole plasma volume is removed, and the cells are dispersed in the remaining plasma to prepare the IL-1Ra enriched composition.

The PRP fraction may be inserted into the second collection tube with glass beads and/or plastic microbeads. When the second tube is preinserted with glass beads only, incubation may proceed at, for example, 37 centigrade with or without 5% CO₂ for 1-24 hours, for example 8-16 hours and the plasma enriched with IL1Ra is collected via disc filter, for example having a pore size of 0.22-0.45 micron to eliminate cells and to provide concentrated plasma with IL-1 Ra at the time of processing, or after 3-12 months from the process time when stored at the appropriate temperature, such as −5 to −20 centigrade.

When a collection tube is preinserted only with polymer microbeads or with polymer micro beads and glass beads, the collecting of the enriched IL-1Ra plasma can be done immediately or up to 1 hour, for example product may be withdrawn after 15-30 minutes via sleeve filter with pore size of 0.22-40 micron and/or via disc filter with preferred pore size of 0.22 micron to 0.45 micron or directly to the syringe.

In one embodiment, whole blood is collected in a glass collection vessel containing density separation gel and glass microbeads, in the absence of anticoagulant, followed by incubating the PRP fraction for 30 minutes to 24 hours. Following incubation, the mixture may be centrifuged at 900 to 2500 g to separate the fractions. To collect IL-1Ra concentrate, the fraction enriched in IL-1Ra may be passed through a 0.2 to 1.0 micron filter separating the microbeads, cells and cellular fractions. Alternatively, the mixture may be centrifuged at 3000-5000 g to remove these components. The resulting plasma composition enriched in IL-1Ra may be used for injection to a patient.

The injection of IL-1Ra enriched plasma can be performed immediately after the above procedures or even after 1-12 months, if the IL-1Ra enriched plasma is stored at an appropriate temperature, such as between −5 to −20° C.

In embodiments, IL-1Ra enriched plasma products prepared according to the methods described herein may be effective as a treatment, such as by injection, for both early and late stage orthopedic pathological conditions and/or to decelerate natural progression of chronic orthopedic diseases. The IL-1Ra enriched plasma products may be effective in patients with early symptoms and those with family history looking for preventive therapies. In embodiments, the products may be used as prophylactic therapy in orthopedic surgery or as a symptom modifier option after injury. Injection intervals can be performed according to severity and reappearance of symptoms.

The autologous IL-1Ra therapy is free of immunological response and provides a non-surgical, minimally invasive therapy without pharmacologic treatment. The IL-1Ra obtained is quite concentrated and substantially free of contamination by erythrocytes and pro-inflammatory white blood cells.

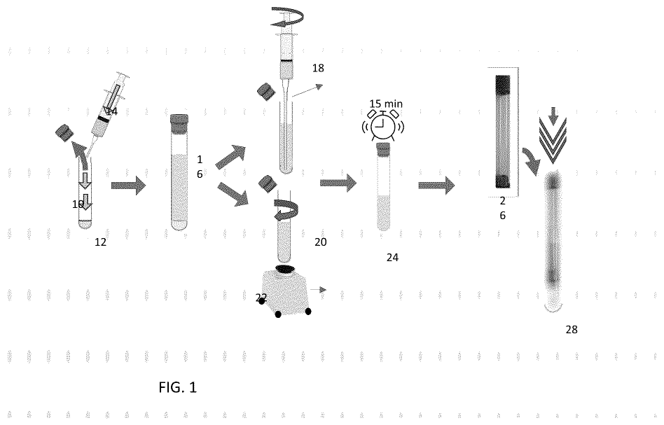

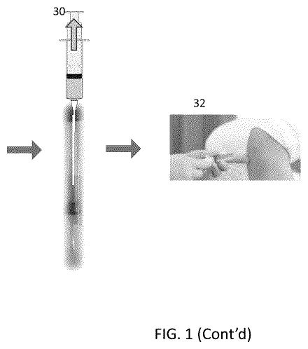

Referring to FIG. 1, a tube 10 may be provided with polymeric microbeads 12 and PRP may be provided to tube 10 from syringe 14. The PRP 20 may be mixed using the blunt needle connected to syringe 18 or using a vortex device 22. At step 24, the mixture is let to stand for about 15 minutes. Thereafter sleeve filter 26 may be inserted toward the bottom of the tube to collect IL-1Ra enriched plasma which may be withdrawn using syringe 30 for use directly with a subject.

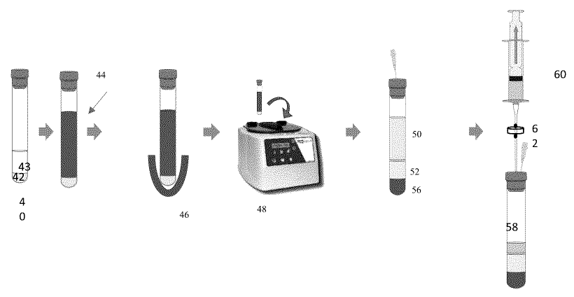

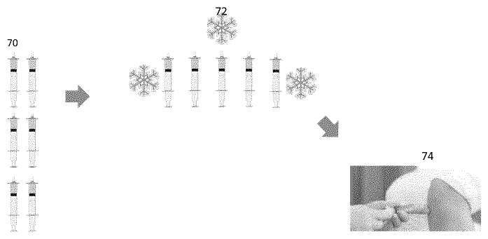

Referring to FIG. 2, in another embodiment, a glass or plastic tube 40 may be provided with glass or polymer microbeads 42 and separation gel 43. Whole blood may be added at step 44 and at step 46 the tube may be either incubated for 4-24 hours or let stand for up to an hour in ambient conditions. Incubation may be followed by centrifugation, for example at at 1000 g to 3000 g to separate plasma fraction 50 from the red blood cells 56 and gel 52. A desired amount of IL-1Ra enriched plasma may be withdrawn from the separated fractions 58 using syringe 60 and a 0.2-1.0 micron disc filter. Aliquots 70 of the IL-1Ra may be collected for dosage to one or more subjects 74 on site or at a later date, provided that suitable storage 72 is provided (−5° C. to −20° C.).

### Example 1

### Tropokine™ IL-1Ra Enriched Plasma Using Microbeads (Suggested Protocol)

### 1. Purpose:

To induce the production of IL-1Ra that is derived from a blood separation platform, e.g., TropoCells® and fused in Tropokine™ P with polyacrylamide (PAA) microbeads.

### 2. Equipment

2.1. 22 mL TropoCells® tubes with gel with anticoagulant
2.2. TropoCells® Vented Needle
2.3. VACU20S
2.4. 10 ml syringe X3
2.5. Disk Filter 40
2.6. Syringe filter 0.22 μm
2.7. Sharp Needle 16G/90 mm
2.8. Two Blunt Needles 100 mm
2.9. Tropokine™ P glass tubes with PAA microbeads
2.10. Sleeve filter
2.11. Centrifuge capable of reaching at least 1500 g
2.12. Vortex machine (optional)

### 3. Procedure

3.1. Blood collection
   3.1.1. Draw blood into a 22 mL TropoCells® tube.
   3.1.2. Invert the tube 2-4 times to mix the blood with the anticoagulant.
   3.1.3. Insert the tube into the centrifuge. Always use a balance tube.
3.2. Centrifugation:
   3.2.1. Spin for 20 min at 1,500 RCF (g).
   3.2.2. Gently take out the tube from the centrifuge and place it in a stand.
3.3. PRP preparation
   3.3.1. Insert the vented needle into the tube rubber stopper.
   3.3.2. Insert a long sharp needle into the tube rubber stopper.
   3.3.3. Attach the Disk Filter to a 10 ml syringe and connect it to the long sharp needle that was inserted into the tube previously (step 3.3.2).
   3.3.4. Withdraw the PPP, leaving behind only 5 ml plasma.
   Note: do not touch the gel with the needle. The needle tip should be above the gel.
   3.3.5. Keep the needle inserted in the tube and only remove the syringe containing the PPP.
   3.3.6. Attach a new 10 ml syringe to the disc filter connected to the long sharp needle.
   3.3.7. Suspend the cells sited on top of the gel to the remaining plasma by inverting the tube ten times.
   3.3.8. Collect the PRP.
   Note: do not touch the gel with the needle. The needle tip should be above the gel.
3.4. IL-1Ra enrichment
   3.4.1. Open the rubber stopper of the 10 mL Tropokine™ P® glass tubes with PAA microbeads and transfer the PRP gradually while gently tilting the tube so that all the microbeads will be soaked with the PRP liquid; use blunt needle connected to syringe to mix well the microbeads with the PRP.
   Note: Ensure all the microbeads are mixed well with the PRP and there are no residues of white powder.
   3.4.2. Close the tube with the rubber stopper and let the tube stand with the PAA microbeads for 15 min at room temperature.
   3.4.3. Remove the rubber stopper and insert the sleeve filter into the tube.
   3.4.4. Push the sleeve filter along the tube.
   Note: Verify that all remaining plasma was extracted and microbeads gel precipitant became whitish.
   3.4.5. Connect the Blunt Needle to a new syringe with a 0.22 μm Syringe filter and insert it into the bottom of the Sleeve Filter to collect the concentrated enriched plasma.
   Optional 1: Use the vortex to mix well the PAA microbeads with the PRP.
   Optional 2: Centrifuge the tube for 5-10 minutes to deposit cells, cellular fractions and microbeads.
   3.4.6. Divide the sample into aliquots and store at −20° C. up to 7 months until analysis with ELISA. Avoid repeated freeze thaw cycles.
3.5. ELISA assessment:
   If IL-1Ra concentration values are required, process the following:
   3.5.1. Perform ELISA with Quantikine Human IL-1ra/IL-1F3 Immunoassay #DRA00B according to the supplier's manual.
Note: In the preliminary studies, the ELISA O.D. wavelength used was 450 nm, without subtracting the O.D. in 540 nm or 570 nm.

As shown in Table 1 below, ELISA assessment of one sample showed a nearly 10-fold increase in the IL-1Ra concentration obtained according to the procedure above, using PAA beads. The “fold” calculation is obtained by dividing the IL-1Ra mass in the final product after treatment by the IL-1Ra mass in the baseline PRP.

TABLE 1

| | IL-1Ra | | | |
|---|---|---|---|---|
| | concentration | Volume | IL-1Ra mass | |
| Sample | (pg/ml) | (mL) | (pg) | Fold |
| T₀ (baseline) | 70.15 | 12 | 841.8 | 1 |
| PAA sterile | 5532 | 1.5 | 8,298 | 9.9 |

### Example 2

Tropokine™ IL-1Ra enriched plasma using glass beads (Suggested Protocol)

### 4. Purpose:

To induce the production of IL-1ra by incubating whole blood in Tropokine™ G tube with glass beads.

### 5. Equipment

5.1. 22 mL or 11 mL TropoCells® tubes with gel
5.2. 22 mL or 11 mL Tropokine™ G tubes with glass beads (optional blood with gel and glass beads)
5.3. Vented Needle
5.4. VACU20S
5.5. 10 ml syringe
5.6. Disk Filter 40 μm
5.7. Syringe filter 0.22 μm
5.8. Sharp Needle 16G/90 mm or 16G/65 mm
5.9. 22 mL or 11 mL TropoCells® tubes with gel (optional)
5.10. Centrifuge capable of reaching at least 2000 g
5.11. Incubator capable of reaching 37° C.

### 6. Procedure

6.1. Blood collection and IL-1Ra enrichment
   6.1.1. Draw blood into 22 mL or 11 mL Tropokine™ G tubes.
   6.1.2. Invert the tube 2-4 times to mix the blood with the glass beads.
   6.1.3. Incubate the tube overnight at 37° C.
6.2. Plasma blood separation:
Option 1
   6.2.1. Following incubation spin the tube for 10 min at 1500-2000 RCF (g).
   6.2.2. Gently take out the tube from the centrifuge and place it in a stand.
   6.2.3. Insert the vented needle into the tube rubber stopper.
   6.2.4. Insert a sharp needle into the tube rubber stopper.
   6.2.5. Attach the 0.22 μm Disk Filter to a 10 mL syringe and connect it to the sharp needle that was inserted into the tube previously (3.2.4)
   6.2.6. Drawn the plasma.
   6.2.7. Divide the sample into aliquots and store at −20° C. up to 7 months until analysis with ELISA. Avoid repeated freeze-thaw cycles.
Option 2—relevant if working with tubes with glass beads only
   6.2.8. Following incubation transfer the blood to TropoCells® tubes with gel without anticoagulant.
   6.2.9. Spin the tube for 10 min at 1,500 RCF (g).
   6.2.10. Insert the vented needle into the tube rubber stopper.
   6.2.11. Insert a sharp needle into the tube rubber stopper.
   6.2.12. Attach the 0.22 μm Disk Filter to a 10 mL syringe and connect it to the sharp needle that was inserted into the tube previously (3.2.11).
   6.2.13. Draw the plasma.
   6.2.14. Divide the sample into aliquots and store at −20° C. up to 7 months until analysis with ELISA. Avoid repeated freeze-thaw cycles.
6.3. ELISA assessment:
If IL-1Ra concentration values are required, process the following:
   6.3.1. Perform ELISA with Quantikine Human IL-1Ra/IL-1F3 Immunoassay #DRA00B according to the supplier's manual.
      Note: In preliminary studies the ELISA O.D. wavelength was 450 nm without subtracting the O.D in 540 nm or 570 nm.

TABLE 2

| | IL-1Ra | | | |
|---|---|---|---|---|
| | concentration | | IL-1ra mass | |
| Sample | (pg/mL) | Volume (mL) | (pg) | Fold |
| t₀ baseline | 10.15 | 6 | 420.9 | 1 |
| Whole blood | 2768 | 5.5 | 15,224 | 36.2 |
| with glass | | | | |
| beads | | | | |
| Whole blood | 1733 | 6 | 10,398 | 24.7 |
| without glass | | | | |
| beads | | | | |

ELISA assessment verified that using the glass beads The “fold” was calculated by dividing the IL-1Ra mass of the baseline after treatment with beads by the IL-1Ra mass in the baseline. Whole blood with glass beads-blood in a glass tube with glass beads Whole blood without glass beads-blood in glass tube without glass beads.

### Example 3—Comparative Effects of Using Glass and Plastic Collection Containers; Polymeric Microbeads; and Glass Beads

Whole blood samples were obtained from four donors and IL-1Ra was measured, reported as concentration (pg/ml) and total mass (pg), at baseline and after processing according the techniques described above, using glass tubes with microbeads only, plastic syringes with microbeads only, glass collection tubes only; glass tube with glass beads and microbeads; and glass tubes with glass beads plus separation gel plus microbeads, with the following results:

TABLE 3

| IL-1Ra concentration (pg/mL) | Donor 1 | Donor 4 | Donor 3 | Donor 2 |
|---|---|---|---|---|
| Baseline (t0) | 197.8 | 235.3 | 131.3 | 152 |
| Glass tube with AC only | 964.4 | 586.6 | 467.3 | 663 |
| Plastic syringe with AC only | 918.9 | 655.7 | 627 | Not Checked |
| Glass tube without AC only | 5109 | 1712 | 795.7 | Not Checked |
| Glass tube with Glass beads + | 888.4 | 666.9 | 685.4 | 663 |
| AC | | | | |
| Glass tube with Glass beads + | 1174 | 668.2 | 708.1 | 744.6 |
| gel + AC | | | | |

TABLE 4

| IL-1Ra mass (pg) | Donor 1 | Donor 4 | Donor 3 | Donor 2 |
|---|---|---|---|---|
| Baseline (t0) | 1,187 | 1,482 | 788 | 1,064 |
| Glass tube with AC only | 6,751 | 4,400 | 3,271 | 4,243 |
| Plastic syringe with AC only | 4,595 | 3,279 | 3,135 | Not Checked |
| Glass tube without AC only | 25,545 | 11,128 | 4,376 | Not Checked |
| Glass tube with Glass beads + | 5,330 | 4,001 | 4,112 | 3,978 |
| AC | | | | |
| Glas tube with Glass beads + | 5,870 | 4,009 | 4,249 | 3,946 |
| gel + AC | | | | |

The volume of the samples is reported in TABLE 5

TABLE 5

| Volumes of samples (ml) | Donor 1 | Donor 4 | Donor 3 | Donor 2 |
|---|---|---|---|---|
| Baseline (t0) | 6 | 6.3 | 6 | 7 |
| Glass tube with AC only | 7 | 7.5 | 7 | 6.4 |
| Plastic syringe with AC only | 5 | 5 | 5 | Not Checked |
| Glass tube without AC only | 5 | 6.5 | 5.5 | Not Checked |
| Glass tube with Glass beads + | 6 | 6 | 6 | 6 |
| AC | | | | |
| Glas tube with Glass beads + | 5 | 6 | 6 | 5.3 |
| gel + AC | | | | |

The fold increase (total mass basis) is reported in TABLE 6

TABLE 6

| Fold of IL-1ra | Donor 1 | Donor 4 | Donor 3 | Donor 2 |
|---|---|---|---|---|
| Baseline (t0) | 1 | 1 | 1 | 1 |
| Glass tube with AC only | 5.7 | 3.0 | 4.2 | 4.0 |
| Plastic syringe with AC only | 3.9 | 2.2 | 4.0 | Not Checked |
| Glass tube without AC only | 21.5 | 7.5 | 5.6 | Not Checked |
| Glass tube with Glass beads + | 4.5 | 2.7 | 5.2 | 3.7 |
| AC | | | | |
| Glas tube with Glass beads + | 4.9 | 2.7 | 5.4 | 3.7 |
| gel + AC | | | | |

### Example 4

In this example, whole blood was collected in a glass tube containing density separation gel and glass microbeads in the absence of anticoagulant. The vessel was incubated for 30 minutes to 24 hours (to prepare different amounts of IL-1Ra in the final product), followed by centrifuging the tube at 1500 g for 10 minutes. The plasma composition enriched in IL-1Ra was obtained directly from the plasma fraction via syringe, passing the mixture through a 0.2 to 1.0 micron disc filter. Table 7 provides shows the average mass in picograms of the IL-1Ra obtained in the samples after different periods of incubation.

TABLE 7
IL-1Ra mass (pg) average

| | | | | | |
|---|---|---|---|---|---|
| Time (hr) | 0 | 0.5 | 1 | 3 | 20 |
| Mass (pg) | 907 | 2,137 | 3,134 | 4,953 | 58,289 |
| Std. Dev. | 527 | 914 | 1664 | 2572 | 33631 |

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A method of preparing a plasma composition enriched in IL-1Ra, comprising:
collecting a PRP fraction in a collection vessel or collection tube containing polymeric microbeads;
mixing the polymeric microbeads with the PRP fraction to form a homogeneous mixture of microbeads and PRP;
allowing the homogeneous mixture to sit for a predetermined period of time;
passing the mixture through a filter, and/or centrifuging the vessel or tube containing the mixture, to separate the microbeads, cells and cellular fractions and to obtain the plasma composition enriched in IL-1Ra.

2. The method according to claim 1, wherein the polymeric microbeads are micron-scale hydrophilic polyacrylamide beads.

3. The method according to claim 2, wherein the polymeric microbeads are mixed with the PRP fraction in a collection tube, and wherein passing the mixture through a filter comprises pressing a sleeve filter into the homogeneous mixture after allowing the homogeneous mixture to sit for about 15 minutes to obtain in the sleeve filter a plasma fraction enriched in IL-1Ra with the beads removed.

4. The method according to claim 3, further comprising removing the plasma fraction enriched in IL-1Ra from the sleeve filter with a syringe while the sleeve filter is inserted in the centrifugation tube.

5. The method according to claim 1, wherein mixing the polymeric microbeads with the PRP comprises mixing with a vortex device or stirring with a needle attached to a syringe.

6. The method according to claim 1, wherein an IL-1Ra concentration, measured in picograms/milliliter, is increased 2.8 to 25.8 fold in the plasma composition enriched in IL-1Ra as compared to a baseline PRP, as determined by ELISA assay

7. The method according to claim 1, wherein total IL-1Ra obtained in the plasma composition, measured in picograms, is increased 2.2 to 21 fold as compared to a baseline.

8. A method of preparing a plasma composition enriched in IL-1Ra, comprising:
collecting a whole blood sample in a centrifugation tube equipped with glass beads, polymeric microbeads or a combination of glass beads and polymeric microbeads;
contacting the whole blood sample with the surface of the beads for a predetermined period of time;
centrifuging the whole blood sample to obtain IL-1Ra enriched plasma;
passing IL-1Ra enriched plasma through a filter to obtain the plasma composition enriched in IL-1Ra.

9. The method according to claim 8, wherein the whole blood sample is collected in a centrifugation tube provided with anticoagulant, separation gel and the glass beads.

10. The method according to claim 8, wherein the whole blood sample is contacted with the surface of the beads for i) 4 to 24 hours at a temperature of 30-40° C. or ii) 5 minutes to 1 hour at room temperature; followed by centrifugation at 1000 g to 3000 g.

11. The method according to claim 8, wherein the IL-Ra enriched plasma is withdrawn from the centrifugation tube via syringe having a disc filter and the IL-Ra enriched plasma is passed through a disc filter to obtain dosage aliquots of IL-Ra enriched plasma.

12. A method of preparing a plasma composition enriched in IL-1Ra, comprising:
collecting whole blood in a glass collection vessel or tube, said vessel or tube containing density separation gel and glass microbeads in the absence of anticoagulant;
incubating the vessel or tube for 30 minutes to 24 hours;
centrifuging the vessel or tube, after incubating, at 900 to 2500 g;
passing the mixture through a 0.2 to 1.0 micron filter, or conducting a second centrifuging step at 3000-5000 g to separate the microbeads, cells and cellular fractions; and
collecting the plasma composition enriched in IL-1Ra.

13. The method according to claim 12, wherein incubation is conducted in an atmosphere containing 5% CO₂.

14. The method according to claim 12, wherein said step of passing the mixture through a 0.2 to 1.0 micron filter comprises passing the mixture through a disc filter directly into a syringe.
